(19) United States
(12) Patent Application Publication
(10) Pub. No.: US 2026/0097106 A1
(43) Pub. Date: Apr. 9, 2026

(54) AVIRULENT LIVE BACTERIAL VACCINES CURED OF PLASMIDS CONTAINING ANTIMICROBIAL RESISTANCE GENES

(71) Applicant: Drs. Koehnk & Feldman, LLP, Jewell, IA (US)

(72) Inventors: Hans Koehnk, Jewell, IA (US); Larry Koehnk, Jewell, IA (US)

(21) Appl. No.: 19/113,283

(22) PCT Filed: Sep. 19, 2023
(86) PCT No.: PCT/US2023/074622

§ 371 Date: Mar. 19, 2025

Related U.S. Application Data
(60) Provisional application No. 63/376,283, filed on Sep. 19, 2022.

(51) Int. Cl. A61K 39/108; A61K 39/00; C12N 15/70

(52) U.S. Cl. CPC A61K 39/0258; C12N 15/70; A61K 2039/522

(57) ABSTRACT

A vaccine comprising a live avirulent strain of bacteria or an immunogenic composition that comprises a live avirulent strain of bacteria, wherein the bacteria is cured of plasmids and/or transposons that contain AMR genes, and a non-conjugative synthetic curing plasmid that cures plasmids and/or transposons that contain AMR genes from such bacteria. A method for producing the live avirulent strain of bacteria or an immunogenic composition that comprises a live avirulent strain of bacteria; a method for treating, preventing, or reducing the severity, duration, or incidence of clinical signs, of a virulent bacterial infection; and a method for preventing, or reducing the risk or incidence of transference of AMR genes are also disclosed.

### STATEMENT REGARDING FEDERALLY SPONSORED RESEARCH & DEVELOPMENT

None.

### FIELD OF THE DISCLOSURE

The field of the disclosure relates generally to immunogenic compositions effective at treating or preventing, or reducing the incidence, severity, frequency, and/or duration of clinical signs of diseases caused by or associated with virulent Escherichia coli (E. coli) bacteria and/or virulent E. coli bacterial infection. More specifically, the disclosure relates to live avirulent E. coli vaccines that have been cured of plasmids and/or transposons that contain antimicrobial resistance (AMR) genes.

### BACKGROUND OF THE DISCLOSURE

Virulent E. coli infections lead to various clinical symptoms and pathogenicity in domesticated agricultural animals, including porcine and bovine varieties, primarily dependent on the serotype of the bacteria and the adherence factors (fimbriae) and toxins, which are often encoded on plasmid DNA (Nagy, Bela, et al., Genetic Diversity among Escherichia coli Isolates Carrying F18 Genes from Pigs with Porcine Postweaning Diarrhea and Edema Disease, Journal of Clinical Microbiology, 37, 5, 1642-45 (1999) (“Nagy, et al.”). Diarrhea and edema are major health problems for porcine and bovine producers that substantially affect swine, poultry and cattle production and results in significant financial losses (Nagy, et al.; Gyles, Carlton L., Overview of Edema Disease, Generalized Conditions, Merck Veterinary Manual, Professional Version, last modified June 2016 (2022) (“Gyles”)). E. coli strains that cause porcine postweaning diarrhea often express an F4 fimbrial adhesin responsible for bacterial attachment to the swine intestine and secrete enterotoxins that cause diarrhea, including the prevalent K88 strain of E. coli (Nagy, et al.; Daudelin, Jean-Fancois, et al., Administration of probiotics influences F4 (K88)-positive enterotoxigenic Escherichia coli Attachment and Intestinal Cytokine Expression in Weaned Pigs, Veterinary Research, 42, 69 (2011)) (“Daudelin, et al.”). Strains that cause edema disease in swine often lack expression of F4, and rather express F18 fimbrial adhesin and produce a Shiga or Shiga-like toxin that causes damage to vascular endothelium, resulting in edema, hemorrhage, intravascular coagulation, and microthrombosis, although certain F18 variants can also be enterotoxigenic (Nagy, et al.; Gyles).

While antimicrobials have enabled successful treatment of infections for years, the overuse and misuse of antimicrobials has led to AMR that health organizations have highlighted as a global health threat (Micoli, Francesca, et al., The Role of Vaccines in Combatting Antimicrobial Resistance, Nature Reviews Microbiology, 19, 287-302 (2021)). Increased concern of AMR gene transfer from the microflora of domestic animals to other bacteria, especially pathogenic bacteria that affect humans, has caused pressure for domestic agricultural producers to consider methods to reduce the risks of transfer of AMR genes (Nagy, et al.). Potential ways organisms containing AMR genes can be exposed to humans include through the human food supply of meat exposed to fecal contamination during slaughter and contaminated feces used as fertilizer. Another potential source of AMR genes with potential for transference is provided with live avirulent vaccines, including E. coli vaccines, that contain AMR genes in their plasmids and/or transposons. However, live bacterial vaccines generally provide a longer lasting immunity than nonviable alternatives. Additionally, orally-administered live bacterial vaccines provide targeted mucosal immunity, as the route of inoculation mimics the route of intestinal E. coli infections in animals. Accordingly, there is a need for live avirulent microbial vaccines that do not contain AMR genes in their plasmids and/or transposons. Such products would provide the important financial and production benefits for swine and cattle producers, and health benefits for swine, cattle, and consumers, without the added risk of transferring AMR genes through plasmids and/or transposons contained in the currently marketed vaccines.

### BRIEF DESCRIPTION OF THE DISCLOSURE

The present disclosure is directed to a vaccine comprising a live avirulent strain of bacteria or an immunogenic composition that comprises a live avirulent strain of bacteria, wherein the bacteria is cured of plasmids and/or transposons that contain at least one AMR gene. In some forms, the bacteria is cured of plasmids and/or transposons that contain 2, 3, 4, 5, 6, 7, 8, 9, 10, 11, 12, 13, 14, 15, 16, 17, 18, 19, or more AMR genes. In some forms, the bacteria is a Gram negative bacteria. In some forms, the bacteria is E. coli. In some forms, the bacteria is an E. coli strain or strains selected from the group consisting of an F18 fimbrial adhesin positive strain, an F4 fimbrial adhesin positive strain, or any combination thereof. In some forms, the bacteria is an E. coli strain that is F18 fimbrial adhesin positive. In some forms, the bacteria is an E. coli strain that is F4 fimbrial adhesin positive. In some forms, the bacteria is cured of at least one virulence gene. In some forms, the virulence gene is astA. In some forms, the bacteria is cured of at least one AMR gene. In some forms, the at least one AMR gene is from an F18 fimbrial adhesin positive strain. In some forms, the at least one AMR gene from the F18 fimbrial adhesin positive strain is selected from the group consisting of cm1A1, sul3, sul2, aph(4)-Ia, aac(3)-IV, aph(6)-Id, aadA2b, aph(3″)-Ib, aadA1, and any combination thereof. In some forms, the cm1A1 gene has at least 90, 91, 92, 93, 94, 95, 96, 97, 98, 99, 99.5, 99.6, 99.7, 99.8, 99.9, or 100% sequence identity or homology with SEQ ID NO. 1. In some forms, the sul3 gene has at least 90, 91, 92, 93, 94, 95, 96, 97, 98, 99, 99.5, 99.6, 99.7, 99.8, 99.9, or 100% sequence identity or homology with SEQ ID NO. 2. In some forms, the sul2 gene has at least 90, 91, 92, 93, 94, 95, 96, 97, 98, 99, 99.5, 99.6, 99.7, 99.8, 99.9, or 100% sequence identity or homology with SEQ ID NO. 3. In some forms, the aph(4)-Ia gene has at least 90, 91, 92, 93, 94, 95, 96, 97, 98, 99, 99.5, 99.6, 99.7, 99.8, 99.9, or 100% sequence identity or homology with SEQ ID NO. 4. In some forms, the aac(3)-IV gene has at least 90, 91, 92, 93, 94, 95, 96, 97, 98, 99, 99.5, 99.6, 99.7, 99.8, 99.9, or 100% sequence identity or homology with SEQ ID NO. 5. In some forms, the cm1A1 gene has at least 90, 91, 92, 93, 94, 95, 96, 97, 98, 99, 99.5, 99.6, 99.7, 99.8, 99.9, or 100% sequence identity or homology with SEQ ID NO. 1. In some forms, the aph(6)-Id gene has at least 90, 91, 92, 93, 94, 95, 96, 97, 98, 99, 99.5, 99.6, 99.7, 99.8, 99.9, or 100% sequence identity or homology with SEQ ID NO. 6. In some forms, the aadA2b gene has at least 90, 91, 92, 93, 94, 95, 96, 97, 98, 99, 99.5, 99.6, 99.7, 99.8, 99.9, or 100% sequence identity or homology with SEQ ID NO. 7. In some forms, the aph(3″)-Ib gene has at least 90, 91, 92, 93, 94, 95, 96, 97, 98, 99, 99.5, 99.6, 99.7, 99.8, 99.9, or 100% sequence identity or homology with SEQ ID NO. 8. In some forms, the aadA1 gene has at least 90, 91, 92, 93, 94, 95, 96, 97, 98, 99, 99.5, 99.6, 99.7, 99.8, 99.9, or 100% sequence identity or homology with SEQ ID NO. 9. In some forms, the at least one AMR gene is from an F4 fimbrial adhesin positive strain. In some forms, the F4 fimbrial positive strain is the K88 strain of E. coli. In some forms, the at least one AMR gene from the F18 fimbrial adhesin positive strain is selected from the group consisting of cm1A1, mdf(A), mef(B), aadA2b, aph(3′)-Ia, aadA1, sul3, and any combination thereof. In some forms, the mdf(A) (or MDFA) gene has at least 90, 91, 92, 93, 94, 95, 96, 97, 98, 99, 99.5, 99.6, 99.7, 99.8, 99.9, or 100% sequence identity or homology with SEQ ID NO. 10. In some forms, the mef(B) gene has at least 90, 91, 92, 93, 94, 95, 96, 97, 98, 99, 99.5, 99.6, 99.7, 99.8, 99.9, or 100% sequence identity or homology with SEQ ID NO. 11. In some forms, the aph(3′)-IA gene has at least 90, 91, 92, 93, 94, 95, 96, 97, 98, 99, 99.5, 99.6, 99.7, 99.8, 99.9, or 100% sequence identity or homology with SEQ ID NO. 12. In some forms, the bacteria is both F18 and F4 adhesin positive. In some forms, the bacteria is cured of at least one AMR gene selected from the group consisting of cm1A1, sul3, sul2, aph(4)-Ia, aac(3)-IV, aph(6)-Id, aadA2b, aph(3″)-Ib, aadA1, mdf(A), mef(B), aadA2b, aph(3′)-Ia, and any combination thereof.

A reference strain of E. coli that is F18 fimbrial positive is provided herein as SEQ ID NO. 13. A reference strain of K88 E. coli is provided herein as SEQ ID NO. 14.

In some forms, the bacteria is an E. coli strain with a genome that has at least 90, 91, 92, 93, 94, 95, 96, 97, 98, 99, 99.1, 99.2, 99.3, 99.4, 99.5, 99.6, 99.7, 99.8, 99.9 or even 100% sequence homology and/or identity with the sequence that can be elaborated by SEQ ID NOS. 17-164, which are the contigs or nodes of an F18 fimbrial positive strain that is cured of at least one AMR gene. In some forms, the F18 fimbrial positive strain is the same strain that is in the Edema Vac vaccine (ARKO Laboratories, Jewell, IA). In some forms, the E. coli F18 fimbrial positive strain that is cured of at least one AMR gene as described above is the same strain as is in the Edema Vac vaccine after it has been cured of the AMR genes after exposure to a curing plasmid, such as the curing plasmid of SEQ ID NO. 15 or 16. In some forms, the contigs or nodes of the cured E coli virus are represented by SEQ ID NOS. 17-164. In some forms, the bacteria is a K88 E. coli strain with a genome that has at least 90, 91, 92, 93, 94, 95, 96, 97, 98, 99, 99.1, 99.2, 99.3, 99.4, 99.5, 99.6, 99.7, 99.8, 99.9 or even 100% sequence homology and/or identity with the sequence of the E. coli virus included in the Entero Vac vaccine, which is a known as a K88 strain. In some forms, the sequence elaborated by the contigs or nodes of SEQ ID NOS: 165-672 represents the Entero Vac strain after it exposure and/or contact with a curing plasmid, such as the curing plasmid of SEQ ID NO. 15 or 16. In preferred forms, the virus of either Edema Vac or Entero Vac has been cured of at least one AMR gene selected from the group consisting of cm1A1, sul3, sul2, aph(4)-Ia, aac(3)-IV, aph(6)-Id, aadA2b, aph(3″)-Ib, aadA1, mdf(A), mef(B), aadA2b, aph(3′)-Ia, and any combination thereof.

The present disclosure is also directed to a non-conjugative synthetic curing plasmid that is designed to cure plasmids and/or transposons that contain AMR genes from a live avirulent strain of bacteria or an immunogenic composition that comprises a live avirulent strain of bacteria, wherein the bacteria or immunogenic composition is a vaccine. In some forms, the non-conjugative synthetic curing plasmid includes incompatibility and/or addiction elements that are the same as the incompatibility and/or addiction elements contained in the plasmids and/or transposons that are cured from the bacteria described above that contains AMR genes prior to being subjected to the curing process described herein. In some forms, the curing plasmid removes at least 1, 2, 3, 4, 5, 6, 7, 8, 9, 10, 11, 12, 13, 14, 15, 16, 17, 18, 19, 20 or more antimicrobial resistance genes that are contained in the plasmids and/or transposons of the bacteria prior to being cured from the bacteria. In some forms, the plasmid comprises at least 1, 2, 3, 4, 5, 6, 7, 8, 9, 10, or more negative growth selection genes. In some forms, the curing plasmid has at least 90, 91, 92, 93, 94, 95, 96, 97, 98, 99, 99.1, 99.2, 99.3, 99.4, 99.5, 99.6, 99.7, 99.8, 99.9 or even 100% sequence homology and/or identity with SEQ ID NO: 15. In some forms, the curing plasmid has at least 90, 91, 92, 93, 94, 95, 96, 97, 98, 99, 99.1, 99.2, 99.3, 99.4, 99.5, 99.6, 99.7, 99.8, 99.9 or even 100% sequence homology and/or identity with SEQ ID NO: 16.

The present disclosure also provides a method for producing a vaccine or immunogenic composition as described herein generally using the methodology described in U.S. Pat. No. 8,877,502. In some forms, the method generally includes transforming a live avirulent bacteria with a curing plasmid designed to cure plasmids and/or transposons that contain antimicrobial resistance genes from the live avirulent bacteria, and selecting for live avirulent bacteria that are cured of plasmids and/or transposons that contain antimicrobial resistance genes. In some forms, the live avirulent bacteria is an E. coli strain with a genome that has at least 90, 91, 92, 93, 94, 95, 96, 97, 98, 99, 99.1, 99.2, 99.3, 99.4, 99.5, 99.6, 99.7, 99.8, 99.9 or even 100% sequence homology with SEQ ID NO: 13 or SEQ ID NO: 14. In some forms, the curing plasmid has at least 90, 91, 92, 93, 94, 95, 96, 97, 98, 99, 99.1, 99.2, 99.3, 99.4, 99.5, 99.6, 99.7, 99.8, 99.9 or even 100% sequence homology to SEQ ID NO: 15 or SEQ ID NO: 16, respectively. In preferred forms, the bacteria has been cured of at least one AMR gene selected from the group consisting of cm1A1, sul3, sul2, aph(4)-Ia, aac(3)-IV, aph(6)-Id, aadA2b, aph(3″)-Ib, aadA1, mdf(A), mef(B), aadA2b, aph(3′)-Ia, and any combination thereof.

The present disclosure further provides methods of treating, preventing, or reducing the severity, duration, or incidence of clinical signs, of a virulent bacterial infection comprising administering the vaccine or immunogenic composition described herein to a mammal. In some forms, the bacteria in the vaccine is an E. coli strain or strains selected from the group consisting of an F18 fimbrial adhesin positive strain, and F4 fimbrial adhesin positive strain, or a combination thereof. In some forms, the mammal is selected from the group of swine and cattle. In some forms, administration of the vaccine or immunogenic composition described herein results in a decrease in the severity, duration, or incidence of clinical signs in comparison to an animal or group of animals that has not received at least one administration of the vaccine or immunogenic composition. In some forms, the decrease is at least 10, 20, 30, 40, 50, 60, 70, 80, 85, 90, 95, or even 100% in comparison to an animal or group of animals that did not receive at least one administration of the vaccine or immunogenic composition described herein.

The present disclosure further provides methods of preventing, or reducing the risk or incidence of, transference of antimicrobial resistance genes from a live avirulent bacterial vaccine to human pathogens generally comprising administering the vaccine or immunogenic composition described herein to a mammal. In some forms, the bacteria in the vaccine is an E. coli strain or strains selected from the group consisting of an F18 fimbrial adhesin positive strain, and F4 fimbrial adhesin positive strain, or a combination thereof. In some forms, the mammal is selected from the group of swine and cattle. In some forms, administration of the vaccine or immunogenic composition described herein results in a decrease in the incidence of transference of antimicrobial resistance genes from a live avirulent bacterial vaccine to human pathogens. In some forms, the decrease is at least 10, 20, 30, 40, 50, 60, 70, 80, 85, 90, 95, or even 100% in comparison to a live avirulent vaccine was not cured of AMR genes as described herein. In preferred forms, the bacteria has been cured of at least one AMR gene selected from the group consisting of cm1A1, sul3, sul2, aph(4)-Ia, aac(3)-IV, aph(6)-Id, aadA2b, aph(3″)-Ib, aadA1, mdf(A), mef(B), aadA2b, aph(3′)-Ia, and any combination thereof.

The present disclosure is also directed to a method of treating, preventing, or reducing the severity, duration, or incidence of clinical signs, of a virulent bacterial infection by vaccination with a live avirulent strain of a bacteria or an immunogenic composition that comprises a live avirulent strain of bacteria, wherein the bacteria is cured of plasmids and/or transposons that contain AMR genes. In preferred forms, the bacteria has been cured of at least one AMR gene selected from the group consisting of cm1A1, sul3, sul2, aph(4)-Ia, aac(3)-IV, aph(6)-Id, aadA2b, aph(3″)-Ib, aadA1, mdf(A), mef(B), aadA2b, aph(3′)-Ia, and any combination thereof.

The present disclosure is also directed to a method of preventing, or reducing the risk or incidence of, transference of AMR genes from a live avirulent strain of a bacteria or an immunogenic composition that comprises a live avirulent strain of bacteria to human pathogens by administering the live avirulent strain of a bacteria or an immunogenic composition that comprises a live avirulent strain of bacteria as a vaccine to animals, wherein the bacteria has been cured of plasmids and/or transposons that contain AMR genes. In preferred forms, the bacteria has been cured of at least one AMR gene selected from the group consisting of cm1A1, sul3, sul2, aph(4)-Ia, aac(3)-IV, aph(6)-Id, aadA2b, aph(3″)-Ib, aadA1, mdf(A), mef(B), aadA2b, aph(3′)-Ia, and any combination thereof.

### DETAILED DESCRIPTION OF THE DRAWING FIGURES

FIG. 1 is a listing of sequence ID Nos. 301-672.

### DETAILED DESCRIPTION

Plasmids with multiple AMR genes considered to be mobile elements were discovered via whole genome sequencing of live avirulent bacterial organisms that are used as vaccines. More specifically, plasmids containing AMR genes were discovered in commercially available live avirulent E. coli vaccines directed to the treatment, prevention, or reduction of severity, duration, or incidence of clinical signs of virulent F18, K88, and in related strains of E. coli, e.g., Escherichia Coli Vaccine, Avirualent Live Culture, Edema Vac, F18 and Escherichia Coli Vaccine, Avirulent Live Culture, Entero Vac, K88 live avirulent E. coli vaccines. The plasmids from these strains of vaccine were analyzed and found to also contain certain incompatibility and addiction elements. Non-conjugative synthetic plasmids containing the same incompatibility and/or addiction elements were designed for the purpose of displacing the AMR-containing natural plasmids through transformation. Methods of curing bacterial cells of plasmids are known in the art, for example, in U.S. Pat. No. 8,877,502, which is incorporated herein by reference in its entirety. In certain aspects, the non-conjugative, synthetic plasmids contain the same incompatibility and addiction elements as the AMR-containing natural plasmids. The synthetic plasmids also contained a positive and negative selection component for the purposes of curing the synthetic plasmid from the E. coli vaccines following displacement of the naturally occurring AMR-containing plasmids. Because the non-conjugative, synthetic plasmid contains the same incompatibility factor as is present in the AMR plasmid, introduction the non-conjugative plasmid into an AMR-containing cell, causes displacement of the existing AMR-containing plasmid. Further, pairing of the anti-toxin encoded by the non-conjugative, synthetic plasmid to the toxin/anti-toxin complex present on the existing AMR ensures survival of the cell containing the non-conjugative, synthetic plasmid following elimination of the AMR-containing plasmid.

Avirulent E. coli vaccine strains previously found in nature and used as licensed vaccines, e.g., Edema Vac F18 and Entero Vac K88 (both ARKO Laboratories, Jewell, IA), produced for administration to swine were propagated and made competent. The E. coli vaccine strains were then transformed with curing plasmids specific to each strain of vaccine via heat shock to allow transformation of the curing plasmids through open calcium channels in the vaccine cells. Transformed colonies were selected and later propagated with negative selection to produce live avirulent strains of E. coli that have been cured of both naturally occurring plasmids containing AMR genes and the specific curing plasmid. The phenotypic characteristics of the vaccines also changed, especially with respect to the loss of AMR resistance associated with AMR genes that were contained in plasmids that were cured from the E. coli vaccine cells. The displacement of the naturally occurring plasmids containing AMR genes and the specific curing plasmid was confirmed by whole genome sequencing.

Avirulent E. coli vaccine strains cured of plasmids and/or transposons that contain AMR genes (e.g., Edema Vac F18-SR and Entero Vac K88-SR produced as disclosed herein) were used to vaccinate swine for the effective prevention, reduction in severity, duration, and/or incidence of infection from a respective F18 or K88 strain of virulent E. coli challenge bacteria.

Advantageously, the methods used for production of live avirulent E. coli vaccines and immunogenic compositions that comprises a live avirulent strain of bacteria, wherein the bacteria has been cured of plasmids and/or transposons that contain AMR genes, and their use as vaccines, can be applied to the production, and use, of other live avirulent bacterial vaccines and immunogenic compositions that comprise live avirulent bacteria.

In some embodiments, the live avirulent strain of bacteria is a either a Gram-positive or Gram-negative bacteria, or a mycoplasma bacteria. In some embodiments, the live avirulent strain of bacteria is a Gram-negative bacteria. In some embodiments, the live avirulent strain of bacteria is a Gram-negative enteric bacteria. In some embodiments, the live avirulent strain of bacteria is a mycoplasma bacteria. In some embodiments, the live avirulent bacteria is a strain of E. coli. In some embodiments, the live avirulent bacteria is a strain of E. coli that expresses F18 fimbrial adhesin. In some embodiments, the live avirulent bacteria is an F18 strain of E. coli that expresses F18 fimbrial adhesin. In some embodiments, the live avirulent bacteria is a strain of E. coli that expresses F4 fimbrial adhesin. In some embodiments, the live avirulent bacteria is a K88 strain of E. coli that expresses F4 fimbrial adhesin. In some embodiments, the live avirulent bacteria is Edema Vac F18 vaccine that has been cured of plasmids and/or transposons that contain AMR genes. In some embodiments, the live avirulent bacteria is Entero Vac K88 vaccine that has been cured of plasmids and/or transposons that contain AMR genes. In preferred forms, the bacteria has been cured of at least one AMR gene selected from the group consisting of cm1A1, sul3, sul2, aph(4)-Ia, aac(3)-IV, aph(6)-Id, aadA2b, aph(3″)-Ib, aadA1, mdf(A), mef(B), aadA2b, aph(3′)-Ia, and any combination thereof.

In some embodiments, the synthetic curing plasmid contains incompatibility features. In some embodiments, the synthetic curing plasmid contains incompatibility features that are different from incompatibility features in the naturally occurring plasmids to be cured from the bacteria. In some embodiments, the synthetic curing plasmid contains incompatibility features that are the same as incompatibility features in the naturally occurring plasmids to be cured from the bacteria. In some embodiments, the synthetic curing plasmid contains incompatibility features that are the same as incompatibility features present in plasmids that naturally occur in Gram-negative bacteria. In some embodiments, the synthetic curing plasmid contains incompatibility features that are the same as incompatibility features present in plasmids that naturally occur in E. coli. In some embodiments, the synthetic curing plasmid contains incompatibility features that are the same as incompatibility features that are present in plasmids in either F18 and/or K88 strains of E. coli. In some embodiments, the synthetic curing plasmid contains incompatibility features that are the same as incompatibility features that are present in plasmids in the Edema Vac F18 and/or the Entero Vac K88 vaccine.

Incompatibility features on the synthetic curing plasmid can include but are not limited to, e.g., the same replicon and/or the same partitioning system as that used by the naturally occurring plasmids to be cured from the bacteria, features that cause a high copy number of the synthetic curing plasmid in relation to the naturally occurring plasmids, iterons that cause downregulation of replication of the naturally occurring plasmids, factors that cause late replication of the naturally occurring plasmids resulting in improper partitioning, and the like.

In some embodiments, the synthetic curing plasmid contains addiction elements. In some embodiments, the synthetic curing plasmid contains addiction elements that are the different from addiction elements in the naturally occurring plasmids to be cured from the bacteria. In some embodiments, the synthetic curing plasmid contains addiction elements that are the same as addiction elements in the naturally occurring plasmids to be cured from the bacteria. In some embodiments, the synthetic curing plasmid contains addiction elements that are the same as addiction elements present in plasmids that naturally occur in Gram-negative bacteria. In some embodiments, the synthetic curing plasmid contains addiction elements that are the same as addiction elements present in plasmids that naturally occur in E. coli. In some embodiments, the synthetic curing plasmid contains addiction elements that are the same as addiction elements that are present in plasmids in either F18 and/or K88 strains of E. coli. In some embodiments, the synthetic curing plasmid contains addiction elements that are the same as addiction elements that are present in plasmids in the Edema Vac F18 and/or the Entero Vac K88 vaccine.

Addiction elements on the synthetic curing plasmid can include but are not limited to, e.g., protein-based toxin-antitoxin systems, restriction modification systems, antisense RNA-regulated systems, and the like.

In some embodiments, the synthetic curing plasmid contains at least one positive and/or negative selection component. In some embodiments, the synthetic curing plasmid contains at least one positive selection component that allows for the selection of bacteria that host the synthetic plasmid that contains the positive selection gene. Examples of positive selection components are antibiotic resistance genes including, but not limited to genes that confer resistance to sodium chlorite, chloramphenicol, tetracycline, neomycin, sulfathiazine, sulfatrim, gentamicin, penicillin, ampicillin, trimethoprim, ciprofloxacin, ceftiofur, clindamycin, danofloxacin, enrofloxacin, florfenicol, Gamithromycin, sulfadimethoxine, spectinomycin, tiamulin, tildipirosin, tilmicosin, Sulphamethoxazole, tulathromycin, and tylosin. In some embodiments, the synthetic curing plasmid contains at least one positive selection component that allows for the selection of bacteria that host the synthetic plasmid that contains the positive selection gene, wherein the positive selection component is different than any positive selection component on the naturally occurring plasmids that are being cured from the bacteria.

In some embodiments, the synthetic curing plasmid contains at least one negative selection component that allows for the selection of bacteria that are not hosting the synthetic plasmid that contains the negative selection gene. Examples of negative selection components include, but are not limited to, toxin-antitoxin systems and SacB counter-selection. Using SacB counter-selection, plating on sucrose medium will select for cells that no longer contain the SacB gene that codes for the enzyme levansucrase, which converts sucrose to a toxic metabolite in Gram-negative bacteria.

In some embodiments, the method of producing a live avirulent strain of bacteria or an immunogenic composition that comprises a live avirulent strain of bacteria, wherein the bacteria is cured of plasmids and/or transposons that contain AMR genes is conducted using methods involving transformation and selection of bacteria. In some embodiments, transformation is conducted using methods including, but not limited to, heat shock or electroporation. In some embodiments, selection is conducted using positive selection methods including, but not limited to, antibiotic resistance or auxotrophy. In some embodiments, selection is conducted using negative selection methods including, but not limited to, toxin-antitoxin systems or SacB counter-selection. In preferred forms, the bacteria has been cured of at least one AMR gene selected from the group consisting of cm1A1, sul3, sul2, aph(4)-Ia, aac(3)-IV, aph(6)-Id, aadA2b, aph(3″)-Ib, aadA1, mdf(A), mef(B), aadA2b, aph(3′)-Ia, and any combination thereof.

As used herein, the term “avirulent bacteria” refers to bacteria that do not cause pathological conditions in an animal when administered to the animal in the amount necessary to elicit the desired immunological response.

An “immunogenic composition” or “vaccine”, in the context of the present disclosure, refers to a composition of matter that comprises at least one live avirulent bacteria, wherein the bacteria is cured of plasmids and/or transposons that contain AMR genes, that elicits and/or enhances an immunological response in the host of a cellular and/or antibody-mediated immune response to the composition or vaccine of interest. Usually, an “immunological response” includes but is not limited to one or more of the following effects: the production or activation of antibodies, B cells, helper T cells, suppressor T cells, and/or cytotoxic T cells and/or γδ T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction in the severity or prevalence of, up to and including a lack of symptoms normally displayed by an infected host, a quicker recovery time and/or a lowered virulent bacterial titer in the infected host.

“Adjuvants” as used herein, can include incomplete Freunds adjuvant (IFA), aluminum-based adjuvants including aluminum hydroxide and aluminum phosphate, Montanide ISA 720, saponins e.g., Quil A, QS-21 (Cambridge Biotech Inc., Cambridge MA), GPI-0100 (Galenica Pharmaceuticals, Inc., Birmingham, AL), water-in-oil emulsion, oil-in-water emulsion, water-in-oil-in-water emulsion. The emulsion can be based in particular on light liquid paraffin oil (European Pharmacopea type); isoprenoid oil such as squalane or squalene oil resulting from the oligomerization of alkenes, in particular of isobutene or decene: esters of acids or of alcohols containing a linear alkyl group, more particularly plant oils, ethyl oleate, propylene glycol di-(caprylate/caprate), glyceryl tri-(caprylate/caprate) or propylene glycol dioleate: esters of branched fatty acids or alcohols, in particular isostearic acid esters. The oil can be used in combination with emulsifiers to form an emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.). John Wiley and Sons, NY, pp51-94 (1995) and Todd et al., Vaccine 15:564-570 (1997). In certain aspects, the adjuvant may be a mucosal adjuvant, such as Seppic IM 1313 VGNST, Seppic Gel 01 PR, or Chitosan.

For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g., vinyls, allyls and other ethylenically unsaturated groups. The unsaturated radicals may themselves contain other substituents, such as methyl. The products sold under the name Carbopol: (BF Goodrich, Ohio, USA) are particularly appropriate. They are cross-linked with an allyl sucrose or with allyl pentaerythritol. Among then, there may be mentioned Carbopol 974P, 934P and 971P. Among the copolymers of maleic anhydride and alkenyl derivative, the copolymers EMA (Monsanto) which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta GA), SAF-M (Chiron, Emeryville CA), monophosphoryl lipid A, Avridine lipid-amine adjuvant, heat-labile enterotoxin from E. coli (recombinant or otherwise), cholera toxin, IMS 1314 or muramyl dipeptide among many others.

Preferably, the adjuvant is added in an amount of about 10 μg to about 10 mg per dose. Even more preferably, the adjuvant is added in an amount of about 100 μg to about 10 mg per dose. Even more preferably, the adjuvant is added in an amount of about 500 μg to about 5 mg per dose. Even more preferably, the adjuvant is added in an amount of about 750 μg to about 2.5 mg per dose. Most preferably, the adjuvant is added in an amount of about 1 mg per dose.

Additionally, the vaccine can include one or more pharmaceutical-acceptable or veterinary-acceptable carriers. As used herein, “a pharmaceutical-acceptable carrier” or “veterinary-acceptable carrier” includes any and all solvents, solutions, dispersion media, coatings, stabilizing agents, diluents, preservatives, antibacterial, antiviral and antifungal agents, isotonic agents, adsorption delaying agents, and the like. Exemplary carriers include liquid carriers (such as water, saline, calcium chloride, culture medium, aqueous dextrose, and glycols) and solid carriers (such as carbohydrates exemplified by starch, glucose, lactose, sucrose, and dextrans, anti-oxidants exemplified by ascorbic acid and glutathione, and hydrolyzed proteins).

In some embodiments, the vaccine of the present disclosure can also comprise the addition of any stabilizing agent, such as for example saccharides, trehalose, mannitol, saccharose, skim milk and the like, to increase and/or maintain product shelf-life and/or to enhance stability.

In some embodiments, the immunological or vaccine composition induces a competitive exclusion effect in the animal receiving the administration thereof. This competitive exclusion effect also results in a decrease in the severity, duration, and/or incidence of clinical signs of E. coli infection.

In some embodiments, the vaccine is first dehydrated. If the composition is first lyophilized or dehydrated, then, prior to vaccination, said composition is rehydrated in aqueous (e.g., saline, PBS (phosphate buffered saline), calcium chloride solution) or non-aqueous solutions (e.g., oil emulsion (mineral oil, or vegetable/metabolizable oil based/single or double emulsion based), aluminum-based adjuvant, carbomer based adjuvant). Non-chlorinated water is especially preferred for reconstitution or rehydration of the vaccine.

In various embodiments, the unit dose or dose size of virus included in a given unit dosage form of vaccine can vary widely, and can depend upon factors such as the age, weight and physical condition of the animal considered for vaccination. Such factors can be readily determined by the clinician or veterinarian employing animal models or other test systems which are well known to the art.

In some embodiments, the vaccine or immunogenic composition of the present disclosure can be administered to an animal in need thereof by any conventional method including, but not limited to, enteral routes of administration (e.g., sublingual, buccal, rectal, and oral routes, including by oral syringe, by gavage, by mixing the vaccine into food or water for consumption), and intranasal routes of administration (e.g., using sprays, pumps, aerosols, and atomizers).

In some embodiments, an effective amount of a vaccine administered to animals provides effective immunity against microbiological infections or a decreased incidence of or severity of clinical signs caused by virulent bacteria and bacterial infections. In some embodiments, the vaccine is administered to susceptible animals, preferably swine or cattle, in one or more doses. In some embodiments, the vaccine may be administered 1 or 2 times at about 1 to about 3 week intervals. In some embodiments a single dose is preferred. Preferably, the first or single administration is performed around 17-18 days of age or older. In other forms, the vaccine or immunogenic composition is administered at weaning or when the animal is about 1 to about 4 weeks of age, assuming a 21 day or older weaning age. If a second administration is desirable or necessary, the second administration is preferably performed about 1 to about 3 weeks after the first administration of the vaccine. In some embodiments, revaccination is performed in an interval of about 1 to about 2 months after administration of any previous vaccination. In some embodiments, administration of subsequent vaccine doses is preferably done on about a 2 month to about an 3 month basis. In another embodiment, animals vaccinated before the age of about 2 to about 6 weeks should be revaccinated. In some embodiments, administration of subsequent vaccine doses is preferably done on about an annual basis.

In some embodiments, the amount of vaccine that is effective depends on the ingredients of the vaccine and the schedule of administration. Typically, an amount of the vaccine contains about 1×10³ to about 1×10⁹ plaque forming units (PFU) per dose. In certain aspects, a dose of vaccine contains about 1×10⁴ to about 1×10⁸ PFU. In certain aspects, a dose of vaccine contains about 1×10⁵ to about 1×10⁷ PFU. In certain aspects, a vaccine may contain at least 1×10⁴ PFU, at least 1×10⁵ PFU, at least 1×10⁶ PFU, at least 1×10⁷ PFU, at least 1×10⁸ PFU, or at least 1×10⁹ PFU per dose.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity.

Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993): Computer Analysis of Sequence Data, Part I, Griffin, A. M., and Griffin, H. G., eds., Humana Press, New Jersey (1994): Sequence Analysis in Molecular Biology, von Heinge, G., Academic Press (1987): Sequence Analysis Primer, Gribskov, M. and Devereux, J., eds., M. Stockton Press, New York (1991); and Carillo, H., and Lipman, D., SIAM J. Applied Math., 48:1073 (1988), the teachings of which are incorporated herein by reference. Preferred methods to determine the sequence identity are designed to give the largest match between the sequences tested.

Methods to determine sequence identity are codified in publicly available computer programs which determine sequence identity between given sequences. Examples of such programs include, but are not limited to, the GCG program package (Devereux, J., et al., Nucleic Acids Research, 12 (1): 387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, MD 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences.

As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

“Sequence homology”, as used herein, refers to a method of determining the relatedness of two sequences. To determine sequence homology, two or more sequences are optimally aligned, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence. Preferably the homologous sequence comprises at least a stretch of 50, even more preferably 100, even more preferably 250, even more preferably 500 nucleotides.

A “conservative substitution” refers to the substitution of an amino acid residue or nucleotide with another amino acid residue or nucleotide having similar characteristics or properties including size, hydrophobicity, etc., such that the overall functionality does not change significantly.

Those of skill in the art will understand that the compositions herein may incorporate known physiologically acceptable, sterile solutions. For preparing a ready-to-use solution for administration, aqueous isotonic solutions, such as e.g., saline or corresponding solutions are readily available. In addition, the immunogenic and vaccine compositions of the present disclosure can include diluents, isotonic agents, stabilizers, or adjuvants. Diluents can include water, saline, dextrose, ethanol, glycerol, and the like. Isotonic agents can include calcium chloride, sodium chloride, dextrose, mannitol, sorbitol, and lactose, among others. Stabilizers include albumin and alkali salts of ethylendiamintetracetic acid, among others. Suitable adjuvants are those described above.

According to a further aspect, the vaccine or immunogenic composition of the present disclosure may further comprise a pharmaceutical acceptable salt, preferably a phosphate salt in physiologically acceptable concentrations. Preferably, the pH of said immunogenic composition is adjusted to a physiological pH, meaning between about 6.5 and 7.5.

The immunogenic compositions described herein can further include one or more other immunomodulatory agents such as, e. g., interleukins, interferons, or other cytokines. In some embodiments, the vaccine or immunogenic composition can also include antibiotics, wherein the antibiotics are limited to those antibiotics for which the live avirulent bacteria contains a gene within its chromosome that confers AMR specific to the included antibiotics.

In some embodiments, the composition is administered with an adjuvant, as described above.

In some embodiments, the composition includes and/or is administered with a veterinary-acceptable carrier, as described above.

In some embodiments, the composition includes and/or is administered with a stabilizer, as described above.

In some embodiments, the composition includes and/or is administered with a pharmaceutical acceptable salt, as described above.

In some embodiments, the composition includes and/or is administered with at least one immunomodulatory agent, as described above.

Their administration modes, dosages and optimum pharmaceutical forms can be determined according to the criteria generally taken into account in the establishment of a treatment adapted to an animal such as, for example, the age or the weight, the seriousness of its general condition, the tolerance to the treatment and the secondary effects noted. Preferably, the vaccine of the present disclosure is administered in an amount that is protective or provides a protective effect against a virulent bacteria, or related strains, that the vaccine was designed to illicit an immune response against.

### DETAILED DESCRIPTION OF THE DISCLOSURE

This written description uses examples to describe the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

The following examples are provided in order to demonstrate and further illustrate certain preferred embodiments and aspects of the present invention and are not to be construed as limiting the scope thereof.

### Example 1: Materials and Methods

Edema Vac® F18 (Edema Vac F18) and Entero Vac® K88 (Entero Vac K88) live avirulent E. coli vaccines (ARKO Laboratories) Max Efficiency™ DH5α E. coli (DH5α E. coli) Competent Cells (sourced from Thermo Fisher (Invitrogen) #18258012), including pUC19 DNA (0.01 μg/ml) and S.O.C. medium: 2% tryptone, 0.5% yeast extract, 10 mM NaCl, 2.5 mM KCl, 10 mM MgCl₂, 10 mM MgSO₄, and 20 mM glucose.

Curing plasmid (with chloramphenicol and sodium chlorite resistance genes) for curing Edema Vac F18 live avirulent E. coli cell vaccine (Source—Chris Thomas, University of Birmingham, UK, SEQ ID NO: 15).

Curing plasmid (with chloramphenicol and sodium chlorite resistance genes) for curing Entero Vac K88 live avirulent E. coli cell vaccine (Source—Chris Thomas, University of Birmingham, UK: SEQ ID NO: 16).

F18 and K88 E. coli reference strains (South Dakota State University) (SEQ ID NOS., 13 and 14, respectively).

Illumina sequencing performed using by Genewiz.

Pigs used for initial F18 study (E. coli susceptible pigs).

Pigs used for F18/K88 study (15 day old pigs from AMVC, LLC, porcine reproductive and respiratory syndrome/Flu negative).

Reference control strains for F18 E. coli (F18 Reference) and K88 E. coli (K88 Reference) (South Dakota State University (SDSU).

F18 monoclonal antibody (RTI, Brookings, South Dakota).

Goat anti-mouse FITC conjugate (MP Biomedicals, Irvine, CA).

Tetracycline (TE 30), Neomycin (NE 30), Sulfathiazine (ST 0.25), Sulfatrim (SXT), and Gentamicin (GM 10) discs.

Liofilchem™ MTS™ Trimethoprim[TM] 0.002-32 μg/mL-MIC test strips (sourced from Fisher Scientific #22-777-761).

Sodium Chlorite (Sigmaaldrich.com, Cat. #244155-5G).

Chloramphenicol.

Ampicillin.

Ciprofloxacin (Alfa Aesar, Thermo Scientific).

Tetracycline HCL.

Sucrose.

Glycerol.

E. coli production media.

F18 production media.

Tris EDTA buffer (TE buffer).

TSB media (Tryptone, Soy, NaCl, dipotassium phosphate (K₂HPO₄), and glucose.

F18 Production Media.

1× LB agar media (Teknova, Cat. #L9105): 1% Tryptone, 0.5% Yeast Extract, 1% Sodium Chloride, 1.5% Agar. Dissolved 40 grams per liter of water, Autoclaved to sterilize.

LB (Miller) broth (Gibco), LB (Miller) broth (Difco), LB broth (Teknova), LB Agar media (Teknova, Cat. #L9105), LB agar containing blood, LB agar containing 25 μg/ml chloramphenicol (Teknova), LB agar containing 100 μg/ml ampicillin, 5% BBA (Brucella Blood Agar) containing 5 μg/ml ciprofloxacin, Blood Agar media, Tryptose Blood Agar Base (Difco, Ref #223220), Donor Bovine Blood/Defibrinated (Quad Five, Prod. #910-500).

Mueller Hinton agar plates.

PureLink™ HiPure Plasmid Maxiprep Kit (Plasmid Maxiprep Kit) (Invitrogen, Cat. #K210006).

Fimbrex K88 agglutination test (Veterinary Laboratories Agency).

DI Water, sterile R/O water, Ultrapure water.

### Example 2: Determination of Minimum Inhibitory Concentration (MIC) for Sodium Chlorite for Entero Vac F18 and Edema Vac K88 Vaccine Strains

Testing for resistance of the live avirulent E. coli strains, Entero Vac K88 and Edema Vac F18, to sodium chlorite was performed by evaluating the minimum inhibitory concentration of sodium chlorite as follows: Serial dilutions of sodium chlorite were prepared from a 100 mM stock solution using sterile TSB media and filter sterilized through 0.22 μm filter. Serially diluted tubes (5 ml each) were inoculated with either Entero Vac K88 or Edema Vac F18 strains of E. coli vaccine by adding 20 μl of 100 dose size of either Entero Vac K88 or Edema Vac F18 vaccine rehydrated in 5 ml TSB media. Positive controls are inoculated TSB media only, negative controls are uninoculated TSB media. All tubes were incubated overnight at 37° C. and turbidity was visualized the following day. Results of the MIC tests are disclosed in Table 1.

TABLE 1

| | | |
|---|---|---|
| NaClO₂ | Edema F18 | Entero K88 |
| Conc. | AMR5 | AMR36 |

| | | | |
|---|---|---|---|
| 10 | mM | Neg | Neg |
| 1 | mM | Neg | Neg |
| 0.5 | mM | Neg | Neg |
| 0.25 | mM | Neg | Neg |
| 0.125 | mM | Neg | Neg |
| 0.0625 | mM | Pos | Pos |
| 0.03125 | mM | Pos | Pos |

| | | |
|---|---|---|
| 0 - Pos Control | Pos | Pos |
| Neg Control | Neg | Neg |

### Example 3: Transformation of DH5a E. coli with AMR5 Plasmid (SEQ ID NO. 17) for Propagation and Amplification

AMR5 plasmids (SEQ ID NO. 17) were reconstituted from a nitrocellulose filter disc in 0.1× TE buffer in RNAse and DNAse free tubes to about 8.5 ng/ml. About 35 ng of AMR5 plasmid DNA (SEQ ID NO. 17) was added to 100 μl of stock DH5a E. coli cells. Transformation was performed by cooling the cells on ice for 30 min, heat shocking at 42° C. for 45 sec, cooling on ice for 2 min, adding 0.9 ml S.O.C. media at room temperature, and incubating for 1 hour at 37° C. on an orbital shaker. Samples were plated using 0.1 ml of serial dilutions of sample (1:10, 1:100, and 1:1,000) on LB agar containing 25 μg/ml chloramphenicol, incubated at 37° C. for 1-2 days, individual colonies picked and streaked on LB agar containing 25 μg/ml chloramphenicol, incubated at 37° C. for 1-2 days, individual colonies picked and sub-cultured in 25 ml LB broth containing 25 μg/ml chloramphenicol, and incubated at 37° C. on orbital shaker at 125 rpm (DH5a AMR5 seeds were prepared by mixing 3.75 ml overnight culture to 1.25 ml sterile glycerol, aliquoted and frozen at −70° C.). Transfer of DH5a AMR5 broth culture to 500 ml of LB broth containing 25 μg/ml chloramphenicol was performed with sterile swab, incubated at 37° C. overnight with magnetic stirrer, pelleted at 4,000×g for 10 min, and resuspended in 200 μl TE buffer (not mentioned here, but is included when processing ARKO2B).

AMR5 plasmid DNA was isolated from DH5a AMR5 cells using a Plasmid Maxiprep Kit.

Positive controls for transformation were performed using DH5α E. coli cells, about 50 pg of pUC19 plasmid DNA, and plating on LB agar containing 100 μg/ml ampicillin.

### Example 4: Transformation of DH5a E. coli with ARKO2B Plasmid (SEQ ID NO. 18) for Propagation and Amplification

ARKO2B plasmids (SEQ ID NO. 16) were reconstituted from a nitrocellulose filter disc in 0.1× TE buffer in RNAse and DNAse free tubes to about 8.5 ng/ml. About 35 ng (10 μl) of ARKO2B plasmid DNA (SEQ ID NO. 16) was added to 100 μl of stock DH5a E. coli cells. Transformation was performed by cooling the cells on ice for 30 min, heat shocking at 42° C. for 45 sec, cooling on ice for 2 min, adding 0.9 ml S.O.C. media at room temperature, and incubating for 30 min at 37° C. on an orbital shaker at 225 rpm. Samples were plated using 0.1 ml of serial dilutions of sample (1:10, 1:100, and 1:1,000) on LB agar containing 25 μg/ml chloramphenicol, incubated at 37° C. for 1-2 days, individual colonies picked and streaked on LB agar containing 25 μg/ml chloramphenicol, incubated at 37° C. for 1-2 days, individual colonies picked and sub-cultured in 3 ml LB broth containing 25 μg/ml chloramphenicol, and incubated at 37° C. overnight on orbital shaker at 125 rpm. (DH5a ARKO2B seeds were prepared by adding sterile glycerol to 10%, aliquoted and frozen at −70° C.). Transferred 1 ml of DH5a ARKO2B broth culture to 25 ml of LB broth containing 25 μg/ml chloramphenicol, and incubated at 37° C. overnight on orbital shaker at 150 rpm. Transferred 10 ml of DH5a ARKO2B broth culture to 600 ml of LB broth containing 25 μg/ml chloramphenicol, incubated at 37° C. overnight on orbital shaker at 150 rpm, pelleted at 4,000×g for 10 min, and resuspended in 200 μl TE buffer.

ARKO2B plasmid DNA was isolated from DH5a ARKO2B cells using a Plasmid Maxiprep Kit following.

Positive controls for transformation were performed using DH5α E. coli cells, about 50 pg (5 μl) of pUC19 plasmid DNA, and plating on LB agar containing 100 μg/ml ampicillin.

### Example 5: Preparation of Competent Edema Vac F18 and Entero Vac K88 Cells

Preparation of Edema Vac F18 subculture was performed by transferring a small amount of Edema Vac F18 vaccine (previously rehydrated in TSB media and frozen) to 25 mL LB (Miller) broth, and incubated at 37° C. on an orbital shaker at 125 rpm overnight. Competent Edema Vac F18 cells (MS112101HM-competent) were prepared by transferring 0.3 ml of the Edema Vac F18 subculture to 25 ml of LB (Miller) broth, incubating at 37° C. on orbital shaker at 125 rpm for 2.25 hours, pelleting at 4500×g at 4° C. in 50 ml centrifuge tube, discarding supernatant, resuspending in 10 ml of 100 mM calcium chloride, re-pelleting at 4500×g at 4° C. in 50 ml centrifuge tube, discarding superatant, resuspending in 2.5 ml solution of 85 mM calcium chloride and 15% glycerol, preparing seeds by aliquoting into cryovials and quick freezing to −70° C. in isopropyl alcohol-dry ice bath, and storing at −70° C. for future use.

Competent Entero Vac K88 cells (ECMS091703HM-competent) were prepared using the same procedure with the exception of replacing the Edema Vac F18 vaccine with the Entero Vac K88 vaccine.

### Example 6: Transformation of Edema Vac F18 and DHSa E. coli Cells With AMR5 Plasmid (SEQ ID NO. 15)

Transformation of competent Edema Vac F18 and DH5α E. coli cells with AMR5 plasmid (SEQ ID NO. 15) was performed separately as follows: Adding 10 ul of AMR5 plasmid prep (SEQ ID NO. 15) to 100 ul Edema Vac F18 competent cells (MS112101HM-competent) or DH5a E. coli cells, incubating on ice for 30 min, heat shocking at 42° C. for 45 sec, replacing to ice for 5 min, adding 0.9 ml S.O.C. media, incubate at 37° C. for 4 hours on orbital shaker at 225 rpm, diluting 1:10 in S.O.C. media, and plating 0.1 ml of serial dilutions on either LB agar containing 1 mM sodium chlorite or LB agar containing 25 μg/ml chloramphenicol. Edema Vac F18 (MS112101HM-AMR5) and DH5α E. coli (DH5α-AMR5) samples transformed with AMR5 plasmid (sodium chlorite resistant) were combined with sterile glycerol to 20% sterile glycerol and stored as seeds at −70° C.

Non-transformed Edema Vac F18 and DH5α E. coli cells and LB agar with no additive were used as controls.

### Example 7: Transformation of Entero Vac K88 and DH5a E. coli Cells with ARKO2B Plasmid (SEQ ID NO. 16)

Transformation of competent Entero Vac K88 and DH5a E. coli cells with AMR5 plasmid (SEQ ID NO. 16) was performed separately as follows: Adding 10 ul of ARKO2B plasmid (SEQ ID NO. 16) prep to 100 ul Entero Vac K88 competent cells (ECMS091703HM-competent) or DH5a E. coli cells, incubating on ice for 30 min, heat shocking at 42° C. for 45 sec, replacing to ice for 2 min, adding 0.9 ml S.O.C. media, incubate at 37° C. for 1 hour on orbital shaker at 225 rpm, diluting 1:10 in LB broth containing 1 mM sodium chlorite, incubating overnight at 37° C., and plating 0.1 ml of serial dilutions on either LB agar containing 1 mM sodium chlorite or LB agar containing 25 μg/ml chloramphenicol. Entero Vac K88 (ECMS091703HM-ARKO2B) and DH5α E. coli (DH5α-ARKO2B) samples transformed with ARKO2B plasmid (sodium chlorite resistant) were combined with sterile glycerol to 10% sterile glycerol and stored as seeds at −70° C.

Non-transformed Entero Vac K88 and DH5α E. coli cells and LB agar with no additive were used as controls.

### Example 8: Selection of Edema Vac F18 Cells Cured of Plasmids that Contain AMR Genes

Selection of Edema Vac F18 cells cured of plasmids that contain AMR genes was performed as follows: thawing, serial diluting, and plating the stored AMR5 transformed Edema Vac F18 cells (MS112101HM-AMR5) on LB agar containing 1 mM sodium chlorite, incubating overnight at 37° C., picking sodium chlorite resistant colonies and tri-streaking on LB agar containing 1 mM sodium chlorite, picking chlorite resistant colonies, inoculating 0.5 ml LB (Miller) broth, transferring 0.1 ml of inoculate to 5 ml of either LB broth or LB broth containing 1 mM sodium chlorite, incubating overnight at 37° C., plating and streaking each on LB agar containing 5% sucrose, incubating overnight at 37° C., picking colonies with growth (MS112101HM-SR), and testing for negative growth in LB broth containing either 25 μg/ml chloramphenicol or 1 mM sodium chlorite versus LB broth without additives. MS112101HM-SR was harvested, mixed with sterile glycerol to 20% glycerol, and stored as seeds at −72° C. The AMR5 plasmid contains the sacB gene, which makes Gram negative organisms, including E. coli, sensitive to sucrose. As such, transformed E. coli that retain the AMR5 plasmid will not grow on media containing 5% sucrose, whereas those cells that lose the plasmid will grow on media containing 5% sucrose.

Plating on LB agar containing blood was used as positive control for initial plating, non-transformed MS112101HM-competent cells were used as control for comparison to the transformed colonies.

AMR testing was performed comparing MS112101HM—SR, MS112101HM-AMR5, and MS112101HM-competent cells cultured on Mueller Hinton agar plates using Sulfatrim (SXT), Tetracycline (TE 30), Neomycin (NE 30), Sulfathiazine (ST 0.25), and Gentamicin (GM 10) discs. Results of the AMR testing are presented in Table 2. Notably, the MS112101HM-SR strain displayed less resistance to SXT, NE 30, ST 0.25, and GM 10, whereas the MS112101HM-AMR5 strain retained resistance to NE 30. Also, all strains maintain tetracycline resistance, which is considered to be incorporated in the bacterial genome.

TABLE 2

| | Ring measurement (radius, mm) | | | | |
|---|---|---|---|---|---|
| Mueller Hinton plates | SXT | TE 30 | NE 30 | ST 0.25 | GM 10 |
| MS112101HM-SR | 16 | 0 | 8 | 15 | 11 |
| MS112101HM-AMR5 | 17 | 0 | 11 | 18 | 11 |
| MS112101HM-competent | 11 | 0 | 7 | 0 | 4 |

A master seed of MS112101HM-SR was produced as follows: subculturing 1 seed of MS112101HM—SR in media 852006041, incubating overnight at 37° C., extracting 220 ml and adding to 24.5 ml glycerol, and storing aliquots at −70° C. (MS060520HM-PCISRAF18).

### Example 9: Selection of Entero Vac K18 Cells Cured of Plasmids that Contain AMR Genes

Selection of Entero Vac K18 cells cured of plasmids that contain AMR genes was performed as follows: subculturing the ARKO2B transformed Entero Vac K88 cells (ECMS091703HM-ARKO2B) in LB broth containing 1 mM sodium chlorite into 3 ml LB broth without sodium chlorite, incubating overnight at 37° C., streaking and picking sodium chlorite resistant colonies, plating and streaking each on LB agar containing 5% sucrose, incubating overnight at 37° C., picking colonies with growth (ECMS091703HM-SR), and testing for negative growth in LB broth containing 1 mM sodium chlorite versus LB broth without additives. ECMS091703HM-SR was sub-cultured on blood agar, harvested, mixed with sterile glycerol to 20% glycerol, and stored as seeds at −72° C. The ARKO2B plasmid contains the sacB gene, which makes Gram negative organisms, including E. coli, sensitive to sucrose.

Plating on LB agar containing blood was used as positive control for initial plating, non-transformed ECMS091703HM-competent cells were used as control for comparison to transformed colonies.

Fimbrex K88 agglutination test was performed on both ECMS091703HM-SR and ECMS091703HM-competent blood agar plates. Strong agglutination reactions were observed for both isolates.

AMR testing was performed comparing ECMS091703HM-SR and ECMS091703HM-competent cells cultured on Mueller Hinton agar plates using Neomycin (NE 30) and Sulfathiazine (ST 0.25) discs. Results of the AMR testing are presented in Table 3. A master plate of ECMS091703HM-SR was produced as follows: subculturing a colony of ECMS091703HM-SR from LB agar containing 5% sucrose on new LB agar containing 5% sucrose, incubating overnight at 37° C., and storing sealed at 2-8° C. Notably, the ECMS091703HM-SR strain displayed less resistance to NE 30 and ST 0.25.

TABLE 3

Ring
measurement
(radius, mm)

| Mueller Hinton plates | NE 30 | ST 0.25 |
|---|---|---|
| ECMS091703HM-SR | 34 | 20 |
| ECMS091703HM-competent | No ring | 13 |
| | 7 | |

### Example 10: Production Preparation of Edema Vac F18 Cells Cured of Plasmids that Contain AMR Genes

Edema Vac F18 cells cured of plasmids that contain AMR genes from master seeds (MS060520HM-PCISRAF18) were used for a production procedure as follows: thawing MS060520HM-PCISRAF18 master seed and adding 21.4 ul to 5 ml F18 production media, transferring 0.25 ml of MS060520HM-PCISRAF18 inoculate to 25 ml F18 production media, incubating at 37° C. on orbital shaker for 22 hours at 200 rpm, adding 6 ml glycerol, aliquoting and storing at −70° C. Production seeds were titrated versus Edema Vac F18 cell control production.

### Example 11: Production Preparation of Entero Vac K88 Cells Cured of Plasmids that Contain AMR Genes

Entero Vac K88 cells cured of plasmids that contain AMR genes from ECMS091703HM-SR colony on LB agar containing 5% sucrose were used for a production procedure as follows: inoculating the ECMS091703HM-SR colony to 60 ml E. coli production media, incubate overnight at 37° C. on orbital shaker at 175 rpm, transferring 60 ml of fermentate to 740 ml of E. coli production media, incubating overnight at 37° C. on orbital shaker at 175 rpm, adding glycerol to 10% concentration, aliquoting and storing at −80° C. (Master seeds of Entero Vac K88 cured of plasmids that contain AMR genes production: MS-K88-030321HM-PC2BSRAK88).

### Example 12: Antibiotic Sensitivity of Edema Vac F18 Cells Cured of Plasmids that Contain AMR Genes

The antibiotic sensitivity of Edema Vac F18 cells cured of plasmids that contain AMR genes (MS060520HM-PCISRAF18) was performed at Iowa State University (ISU) (Accession: 2020076670) with comparison to MS112101HM-competent cells (see Tables 4 and 5). Results indicate an increase in antibiotic sensitivity for the cured MS060520HM-PCISRAF18 cells over the MS112101HM-competent cells (including loss of antibiotic resistance to at least ampicillin, tetracycline, florfenicol, gentamicin, and sulfadimethoxine; however as shown, the cured MS060520HM-PCISRAF18 cells retained resistance to ampicillin, clindamycin, penicillin, tetracycline, tiamulin, and tilmicosin.

TABLE 4

| | F18 Pre-Cured | F18 Cured |
|---|---|---|
| | cells | vaccine |
| | (MS112101HM- | (MS060520HM- |
| | competent) | PCISRAF18) |
| E. COLH | *Int/MIC | *Int/MIC |
| Ampicillin | R/>16.0000 | R/>16.0000 |
| Ceftiofur | S/0.5000 | S/<=0.2500 |
| Clindamycin | R/>16.0000 | R/>16.0000 |
| Enrofloxacin | S/<=0.1200 | S/<=0.1200 |
| Florfenicol | R/8.0000 | S/2.0000 |
| Gentamicin | R/16.0000 | S/<=1.0000 |
| Neomycin | S/<=4.0000 | S/<=4.0000 |
| Penicillin | R/>8.0000 | R/>8.0000 |
| Sulfadimethoxine | R/>256.0000 | S/<=256.0000 |
| Spectinomycin | NI/>64.0000 | NI/16.0000 |
| Tetracycline | R/>8.0000 | R/>8.0000 |
| Tiamulin | R/32.0000 | R/>32.0000 |
| Tilmicosin | R/>16.0000 | R/>16.0000 |
| Trimethoprim/ | NI/<=2.0000 | NI/<=2.0000 |
| Sulphamethoxazole | | |
| Tylosin | NI/>32.0000 | NI/>32.0000 |
| (Tartrate/Base) | | |
| Danofloxacin | NI/<=0.1200 | NI/<=0.1200 |
| Tulathromycin | NI/<=8.0000 | NI/<=8.0000 |
| Gamithromycin | NI/4.0000 | NI/4.0000 |
| Tildipirosin | NI/4.0000 | NI/4.0000 |
| Positive Control | OK/3413.3333 | OK/3450.3333 |

TABLE 5

| | F18 Cured vaccine |
|---|---|
| | (MS060520HM- |
| | PCISRAF18) |
| E. COS | *Int/MIC |
| Ampicillin | S/4.0000 |
| Ceftiofur | S/0.5000 |
| Clindamycin | R/>16.0000 |
| Enrofloxacin | S/<=0.1200 |
| Florfenicol | I/4.0000 |
| Gentamicin | S/<=1.0000 |
| Neomycin | S/<=4.0000 |
| Penicillin | R/>8.0000 |
| Sulfadimethoxine | S/<=256.0000 |
| Spectinomycin | NI/<=8.0000 |
| Tetracycline | S/1.0000 |
| Tiamulin | R/>32.0000 |
| Tilmicosin | R/>16.0000 |
| Trimethoprim/ | NI/<=2.0000 |
| Sulphamethoxazole | |
| Tylosin | NI/>32.0000 |
| (Tartrate/Base) | |
| Danofloxacin | NI/<=0.1200 |
| Tulathromycin | NI/<=8.0000 |
| Gamithromycin | NI/8.0000 |
| Tildipirosin | NI/8.0000 |
| Positive Control | OK/3493.66 |

Further evaluation of tetracycline sensitivity of cured MS060520HM-PCISRAF 18 cells was performed as follows: inoculating 10 ml of TSB media with MS060520HM-PCISRAF18 cells, incubating at 37° C. for 22 hours on orbital shaker at 150 rpm, preparing 1:1 serial dilutions of 5 ml tetracycline HCL in TSB media (from stock of 640 mg tetracycline HCl in 5 ml ultrapure water), inoculating the serial dilutions with 10 ul of the fermentate. Results, presented in Table 6 confirm that the MS060520HM-PCISRAF18 cells retain tetracycline resistance and has inhibited growth at 128 μg/ml of tetracycline HCl in TSB media.

TABLE 6

| Tet HCl | Turbidity 8 | Turbidity 24 |
|---|---|---|
| Conc. | hours post | hours post |
| (μg/ml) | inoculation | inoculation |
| 128 | Neg | Pos |
| 64 | Slight | Pos |
| 32 | Pos | Pos |
| 16 | Pos | Pos |
| 8 | Pos | Pos |
| 4 | Pos | Pos |
| 2 | Pos | Pos |
| 0 (control) | Pos | Pos |

Comparison of tetracycline resistance between Edema Vac F18 cells cured of plasmids that contain AMR genes (MS060520HM-PCISRAF18) and Entero Vac K88 cells cured of plasmids that contain AMR genes (MS-K88-030321HM-PC2BSRAK88) was performed as follows: preparing subcultures of MS060520HM-PCISRAF18 and MS-K88-030321HM-PC2BSRAK88 on blood agar containing 16 μg/ml tetracycline HCl, incubating for 18 hours at 37° C., and observing haemolytic growth. Results confirmed tetracycline resistance for MS060520HM-PCISRAF18, while MS-K88-030321HM-PC2BSRAK88 did not display any tetracycline resistance.

### Example 13: Antibiotic Sensitivity of Entero Vac K88 Cells Cured of Plasmids that Contain AMR Genes

The antibiotic sensitivity of Entero Vac K88 cells cured of plasmids that contain AMR genes (MS-K88-030321HM-PC2BSRAK88) was performed at Iowa State University (ISU) with comparison to ECMS091703HM-competent cells. Results indicated an increase in antibiotic sensitivity between the competent cells and the cured cells as presented in Table 7. Notably the cured MS-K88-030321HM-PC2BSRAK88 cells were more sensitive to florfenicol, neomycin, sulfadimethoxine, and tetracycline.

TABLE 7

| | | K88 Cured |
|---|---|---|
| | K88 Pre-Cured | vaccine |
| | cells | (MS-K88- |
| | (ECMS091703HM- | 030321HM- |
| | competent) | PC2BSRAK88) |
| E. COS | *Int/MIC | *Int/MIC |
| Ampicillin | S/4.0000 | S/4.0000 |
| Ceftiofur | S/0.5000 | S/0.5000 |
| Clindamycin | R/>16.0000 | R/>16.0000 |
| Danofloxacin | NI/<=0.1200 | NI/<=0.1200 |
| Enrofloxacin | S/<=0.1200 | S/<=0.1200 |
| Florfenicol | R/8.0000 | I/4.0000 |
| Gamithromycin | NI/>8.0000 | NI/8.0000 |
| Gentamicin | S/<=1.0000 | S/<=1.0000 |
| Neomycin | R/16.0000 | S/<=4.0000 |
| Penicillin | R/>8.0000 | R/>8.0000 |
| Sulfadimethoxine | R/>256.0000 | S/<=256.0000 |
| Spectinomycin | NI/>64.0000 | NI/<=8.0000 |
| Tetracycline | R/>8.0000 | S/1.0000 |
| Tiamulin | R/>32.0000 | R/>32.0000 |
| Tildipirosin | NI/8.0000 | NI/8.0000 |
| Tilmicosin | R/>16.0000 | R/>16.0000 |
| Trimethoprim/ | NI/<=2.0000 | NI/<=2.0000 |
| Sulphamethoxazole | | |
| Tulathromycin | NI/32.0000 | NI/<=8.0000 |
| Tylosin | NI/>32.0000 | NI/>32.0000 |
| (Tartrate/Base) | | |
| Positive Control | OK/3462.3333 | OK/3493.66 |

### Example 14: Analysis of Edema Vac F18 Cells Cured of Plasmids that Contain AMR Genes and Entero Vac K88 Cells Cured of Plasmids that Contain AMR Genes

Agglutination testing was performed on subcultures of Entero Vac K88 competent cells (ECMS091703HM-competent) and production of Entero Vac K88 cells cured of plasmids that contain AMR genes (MS-K88-030321HM-PC2BSRAK88) as follows: ECMS091703HM-competent cells and MS-K88-030321HM-PC2BSRAK88 cured cells were sub-cultured and tri-streaked on blood agar from master seeds of each, incubated overnight at 37° C. Agglutination was confirmed for each sample versus positive and negative control samples.

Reference control strains for F18 E. coli (F18 Reference: SEQ ID NO. 13) and K88 E. coli (K88 Reference; SEQ ID NO. 14) were each sub-cultured on blood agar, incubated overnight at 37° C., and streaked for isolation. A colony of each was transferred to 15 ml TSB media, incubated overnight at 37° C. on orbital shaker at 175 rpm, added to 1.5 ml glycerol, aliquoted, and stored at −80° C.

Goat anti-mouse FITC conjugate was reconstituted with 2 ml ultrapure water, diluted to 1:10, aliquoted and stored at either 2-8° C. or at −80° C.

Results were positive.

### Example 15: Sequencing of Vaccines, Curing Plasmids, and Cured Vaccines

Sequencing of the genetic composition of the Edema Vac F18 vaccine, the Entero Vac K88 vaccine, the AMR5 plasmid for curing Edema Vac F18 vaccine (SEQ ID NO: 15), the ARKO2B plasmid for curing Entero Vac K88 vaccine (SEQ ID NO: 16), the Edema Vac F18 cells cured of plasmids containing AMR genes from production run (MS060520HM-PCISRAF18) (elaborated by the nodes or contigs of SEQ ID NOS: 17-164), and the Entero Vac K88 cells cured of plasmids containing AMR genes from production run (MS-K88-030321HM-PC2BSRAK88) (elaborated by the contigs or nodes of SEQ ID NOS: 165-672) was performed by Genewiz using Illumina sequencing.

Virulence factor analysis was performed for the Edema Vac F18 cells cured of plasmids containing AMR genes from production run (MS060520HM-PCISRAF18), and the Entero Vac K88 cells cured of plasmids containing AMR genes from production run (MS-K88-030321HM-PC2BSRAK88). The results showed that one of the virulence genes was removed.

A sequence search was performed for the Edema Vac F18 cells cured of plasmids containing AMR genes from production run (MS060520HM-PCISRAF18) (elaborated by the contigs or nodes of SEQ ID NOS: 17-164), the Entero Vac K88 cells cured of plasmids containing AMR genes from production run (MS-K88-030321HM-PC2BSRAK88) (elaborated by the contigs or nodes of SEQ ID NOS: 165-672), the AMR5 plasmid for curing Edema Vac F18 vaccine (SEQ ID NO: 15), and the ARKO2B plasmid for curing Entero Vac K88 vaccine (SEQ ID NO: 16). The results showed that AMR genes were eliminated.

What is claimed is:

1. A bacterial vaccine comprising a live avirulent bacteria, wherein the bacteria is cured of plasmids and/or transposons that contain antimicrobial resistance genes.

2. The vaccine of claim 1, wherein the bacteria is a Gram negative bacteria.

3. The vaccine of claim 1, wherein the bacteria is E. coli.

4. The vaccine of claim 1, wherein the bacteria is cured of plasmids that contain antimicrobial resistance genes.

5. The vaccine of claim 1, wherein the bacteria has been cured of at least one AMR gene selected from the group consisting of cm1A1, sul3, sul2, aph(4)-Ia, aac(3)-IV, aph(6)-Id, aadA2b, aph(3″)-Ib, aadA1, mdf(A), mef(B), aadA2b, aph(3′)-Ia, and any combination thereof.

6. The vaccine of claim 1, wherein the bacteria is an E. coli strain or strains selected from the group consisting of an F18 fimbrial adhesin positive strain, and F4 fimbrial adhesin positive strain, or any combination thereof.

7. The vaccine of claim 1, wherein the bacteria is an E. coli strain that is F18 fimbrial adhesin positive.

8. The vaccine of claim 1, wherein the bacteria is an E. coli strain that is F4 fimbrial adhesin positive.

9. The vaccine of claim 1, wherein the bacteria is an E. coli strain with a genome that has at least 90% sequence homology with an E. coli strain that has been cured of at least one AMR gene selected from the group consisting of cm1A1, sul3, sul2, aph(4)-Ia, aac(3)-IV, aph(6)-Id, aadA2b, aph(3″)-Ib, aadA1, mdf(A), mef(B), aadA2b, aph(3′)-Ia, and any combination thereof.

10. A non-conjugative synthetic curing plasmid comprising incompatibility and/or addiction elements that are the same as the incompatibility and/or addiction elements contained in the plasmids and/or transposons that are cured from the bacteria of claim 1.

11. The curing plasmid of claim 10, wherein the curing plasmid comprises at least one antimicrobial resistance gene that is not contained in the plasmids and/or transposons that are cured from the bacteria of claim 1.

12. The curing plasmid of claim 11, wherein the antimicrobial resistance gene is selected from the group consisting of cm1A1, sul3, sul2, aph(4)-Ia, aac(3)-IV, aph(6)-Id, aadA2b, aph(3″)-Ib, aadA1, mdf(A), mef(B), aadA2b, aph(3′)-Ia, and any combination thereof.

13. The curing plasmid of claim 10, wherein the curing plasmid comprises at least one antimicrobial resistance gene that is not contained in the plasmids and/or transposons that are cured from the bacteria of claim 1, and wherein the plasmid comprises at least one negative growth selection gene.

14. The curing plasmid of claim 13, wherein the antimicrobial resistance gene is selected from the group consisting of cm1A1, sul3, sul2, aph(4)-Ia, aac(3)-IV, aph(6)-Id, aadA2b, aph(3″)-Ib, aadA1, mdf(A), mef(B), aadA2b, aph(3′)-Ia, and any combination thereof.

15. A method of treating, preventing, or reducing the severity, duration, or incidence of clinical signs, of a virulent bacterial infection comprising producing the vaccine of claim 1, and administering the vaccine to a mammal.

16. The method of claim 15, wherein the bacteria in the vaccine is an E. coli strain or strains selected from the group consisting of an F18 fimbrial adhesin positive strain, and F4 fimbrial adhesin positive strain, or a combination thereof, and wherein the mammal is selected from the group of swine and cattle.

17. The method of claim 15, wherein the bacteria in the vaccine has been cured of at least one antimicrobial resistance gene selected from the group consisting of cm1A1, sul3, sul2, aph(4)-Ia, aac(3)-IV, aph(6)-Id, aadA2b, aph(3″)-Ib, aadA1, mdf(A), mef(B), aadA2b, aph(3′)-Ia, and any combination thereof.

18. A method of preventing, or reducing the risk or incidence of, transference of antimicrobial resistance genes from a live avirulent bacterial vaccine to human pathogens comprising producing the vaccine of claim 1, and administering the vaccine to a mammal.

19. The method of claim 18, wherein the bacteria in the vaccine is an E. coli strain or strains selected from the group consisting of an F18 fimbrial adhesin positive strain, and F4 fimbrial adhesin positive strain, or a combination thereof, and wherein the mammal is selected from the group of swine and cattle.

20. The method of claim 18, wherein the at least one antimicrobial resistance gene is selected from the group consisting of cm1A1, sul3, sul2, aph(4)-Ia, aac(3)-IV, aph(6)-Id, aadA2b, aph(3″)-Ib, aadA1, mdf(A), mef(B), aadA2b, aph(3′)-Ia, and any combination thereof.
